(19) United States
(12) Patent Application Publication
(10) Pub. No.: US 2017/0049621 A1
(43) Pub. Date: Feb. 23, 2017

(54) PROCESS AND SYSTEM FOR MATERIAL REMOVAL

(71) Applicant: Carl Zeiss Meditec AG, Jena (DE)

(72) Inventors: Manfred Dick, Gefell (DE); Jens Elbrecht, Jena (DE); Eckhard Schroeder, Eckental (DE); Bernhard Seitz, Jena (DE)

(21) Appl. No.: 15/262,826

(22) Filed: Sep. 12, 2016

Related U.S. Application Data
(63) Continuation of application No. 12/380,403, filed on Feb. 26, 2009.
Application No. 12/380,403 is a continuation of application No. 10/480,883, filed on Jun. 28, 2004, now abandoned.

(30) Foreign Application Priority Data
Jun. 15, 2001 (DE) 101 29 650.9

(51) Int. Cl. A61F 9/008

(52) U.S. Cl. CPC A61F 9/00802; A61F 2009/00872; A61B 2018/00017

(57) ABSTRACT

An arrangement including an air conveying device and a suction device guiding an air flow across a site for a duration of a process. The direction of air flowing towards the surface and a tangent to the surface at the ablation site intersect at an angle. A flow velocity of the air flow is controlled. A direction of air flowing away from the surface and the tangent at the site intersect. The flow velocity is selectable, within a specified range. A flow velocity is variable.

### CROSS-REFERENCE TO RELATED APPLICATIONS

This application is a continuation of application Ser. No. 12/380,403, filed Feb. 26, 2009, entitled “PROCESS AND SYSTEM FOR MATERIAL REMOVAL”, which in turn is a continuation of application Ser. No. 10/480,883, filed Jun. 28, 2004, entitled “PROCESS AND SYSTEM FOR MATERIAL REMOVAL”, now abandoned, which is a National Stage Entry of PCT/EP02/01651, filed Jun. 5, 2002, entitled “METHOD AND SYSTEM FOR THE REMOVAL OF MATERIAL”, which claims priority to German Application No. 101 29 650.9, filed Jun. 15, 2001, entitled “METHOD AND SYSTEM FOR THE REMOVAL OF MATERIAL”, each of which is hereby fully incorporated herein by reference.

### TECHNICAL FIELD

The invention relates generally to an air conveying device and a suction device guiding an air flow across a site for the duration of a process. More specifically, the invention relates to conveying air flow in a direction of flow towards the surface and the tangent to the surface with an air flow velocity and a direction of air flowing away from the surface and a relative to a tangent to the surface.

### BACKGROUND

Processes and systems to remove material from the surface of an object using a laser beam directed at the surface are well known. Among these are processes and systems in which the laser beam is scanned over the surface, in the process removing the material in a defined manner and changing the geometry of the object in a controlled fashion.

In the process, the laser energy must be applied around the point of removal without causing significant thermal damage to the area, particularly in soft, temperature-sensitive materials. This is especially important when the material to be removed is very moist and if it is to be prevented from drying out as a result of the energy input in order to prevent the material characteristics or the conditions for material removal, such as removal rate, from changing in undesirable ways during the removal process.

For example, this is the case when the surface curvature of a synthetic contact lens is to be enlarged or reduced to correct the vision of a human eye with the help of the contact lens.

However, in the case of treatment of dead or living biological tissue such as cartilage, tooth enamel or even in eye surgery in the shaping of the cornea (photorefractive keratotomy), not only does care need to be taken during the shaping process, but also the characteristics of the material left in place must be maintained.

Of particular importance in the defined removal of material in the applications mentioned above is the maintenance of climatic conditions in the environment surrounding the point of removal during the length of time of material removal. These conditions are mainly determined by the temperature and humidity at the material surface and in its immediate vicinity.

However, of further importance in the defined removal of material is the continuity of the energy input into the material. Since the laser radiation traverses the open atmosphere or perhaps a protective gas along the path between a radiating optic and the point of removal, it is possible for the by-products arising from the removal of the material, such as smoke or material particles, to impair the atmosphere in the direct vicinity of the point of removal, and in the process to weaken the intensity of the laser radiation in undefined ways by passing through the laser beam.

From U.S. Pat. No. 5,344,418, a system is known in which flow channels are provided near the discharge opening for a laser beam issued from a device designed for material removal. A gas or air stream is directed from these channels to the point of removal as the material removal is occurring, thus enabling the smoke and material particles to be blown away from the point of material removal. However, a disadvantage of this system is that the gas or air stream passes over the material surface at the point of removal, which results in the destruction of any existing film of moisture on the surface and thus taking away its protective function, as well as the film's being dried out to an unacceptable degree, particularly for moist, very hygroscopic materials, thereby subjecting the hydration in the material to an undesired influence during the removal process.

In a device described in U.S. Pat. No. 5,181,916, the contamination, such as smoke or material particles, is not blown away, but is sucked off using a gas stream. To this end, a suction opening is arranged concentrically around a mouth of a device from which the laser beam exits and which is directed to the point of removal.

Here as well, the gas flowing across the point of treatment results in both the moisture at the material surface being drawn off as well as the material drying out at least next to the point of removal.

In DE 100 20 522 A1, a system for suctioning off by-products during ablation of biological tissue is described. Here, the laser beam is directed through a tubular channel onto the tissue and the by-products are sucked off into the channel. An air stream that flows in the opposite direction to the laser radiation is produced inside the channel, wherein the suctioned air does not come from the area surrounding the point of removal, but flows from the feed openings located next to the mouth of the channel. In this way, the material surface is not passed over by the air stream, thus preventing it from drying out. Also, the air stream is directed radially outward from the center of the channel in which the laser beam runs, so that smoke and material particles are kept away from the center and thus from the laser beam, thus preventing the intensity of the laser beam from being influenced by this kind of contamination in an undesired way.

Nevertheless, this method has still not succeeded in protecting the material removal process using laser energy, in particular where very fine treatment of surfaces is performed, from all environmental influences. The removal conditions are still influenced by temperature and humidity at the point of removal, which change during the removal process, despite the measures cited above. In order to attain a higher precision during the shaping via material removal, the need still remains of reducing these types of influences.

### SUMMARY

With this in mind, the purpose of this invention is to maintain the climatic environmental conditions at the point of removal during the entire time of the removal process, while maintaining or even improving the known measures to keep the laser beam cross section free from contamination.

According to the invention and in a process of the type mentioned above, the temperature and/or the humidity at the point of removal and/or in its direct vicinity is held essentially constant by means of a gas that flows in a prescribed direction across the point of removal for the duration of the removal process. In the process, the gas has a prescribed temperature, a prescribed humidity content and/or a prescribed flow velocity.

In a first embodiment of the invention, an air stream with a constant temperature, a constant humidity content and a constant flow velocity is passed over the point of removal for the entire duration of the removal process. This removes excess heat energy by using the air as a transport medium and by appropriately selecting the temperature of the air directed at the point of removal to be below the required temperature at the point of removal. Vice versa, the air directed at the point of removal has a relatively high relative humidity, thus ensuring an influx of moisture and counteracting the tendency of drying out at the material surface and inside the material. For example, the air stream can be directed at the point of removal with a temperature of 37° and a relative humidity of 100% at a flow velocity of approximately 0.5 m/s.

Depending on the characteristics of the material to be treated, it can prove to be favorable if the air stream is passed over the point of removal within a temperature range of −20° to 30° C., a relative humidity in the range of 0-100% and a flow velocity in the range of 1 m/s to 10 m/s. A frequently preferred variation is comprised of flowing air with a temperature of −8° C. and a relative humidity of 80% at approximately 3 m/s across the point of removal.

This makes it possible to hold the climatic conditions constant during the removal within a relatively narrow range. If, for example, during the removal process, an energy load of approximately 0.5 watts is scanned continuously into the material, the major portion of this power will indeed be used for the ablation, but a considerable portion of it will be converted to thermal energy, which, however, is for the most part removed according to the process of the invention so that, as already described, steady-state equilibrium is essentially maintained.

Furthermore, the scope of the invention also encompasses the case where the flow velocity and the quantity of the air stream are prescribed as a function of the pulse repetition frequency of the laser radiation used for the ablation such that the tissue ablated during an impulse sequence can be removed along with the air stream during the time that passes up to the beginning of the next impulse sequence. This is, for example, possible at a pulse repetition frequency of 1 kHz and a surface area treated at the point of removal of 8 mm², with a flow velocity of approx. 8 m/s, wherein the air volume should be approximately 40 cm³/s. In this case, a hose with an approximately 8 mm diameter can be used.

In a preferred embodiment of the invention, an air stream is passed over the point of removal with a constant temperature and a constant humidity content, but with increasing flow velocity through the duration of the removal process. Here, as well, the air stream can have a temperature of 37° and a relative humidity of approx. 100%, for example. However, at the beginning of the removal, the flow velocity is approx. 0.2 m/s and as the removal proceeds is increased to up to 10 m/s. In this way, excess thermal energy can be removed even in the case of higher energy inputs.

It is also within the scope of the invention to feed the air stream at constant flow velocity across the point of removal, but in contrast to lower the temperature of the flowing air and or to increase its relative humidity during the removal process. To this end, for example, the flow velocity of the air throughout the entire removal process can be 0.5 m/s, whereas the air temperature changes within a range of approx. 42° C. at the beginning to approx. 10° C. at the end of the removal process and the humidity changes from approx. 80% at the beginning to 100% at the end of the removal process. This results in even better results in establishing temperature and humidity equilibrium between the material and the climatized environment at the material surface than during constant temperature and humidity, resulting in even more reproducible conditions during shaping.

In embodiment variations of this type, it is possible to make the changes of temperature and/or humidity during the removal process both continuously and discontinuously using prescribed time functions.

Alternatively, it is also conceivable to cause the change in temperature, relative humidity and/or flow velocity of the air to be a function of temperature and/or humidity values that are directly measured, evaluated and used as control parameters for changes made continuously in the air stream at or in the vicinity of the point of removal during the removal process. Thus, for example, a continuous measurement of temperature and humidity at the point of removal provides information that can be used to lower the temperature of the air or to increase its humidity or even to change the flow velocity in order to actively influence, in an appropriate manner, the maintenance of the climatic conditions at the point of removal continuously.

In connection with the measures to maintain the environmental conditions cited above, the direction of the air stream is also constantly such that the by-products resulting from the removal, such as smoke and material particles, are collected by the air stream and removed with the flowing air from the point of removal without passing through the laser beam directed at the point of removal.

Reference is made expressly that the invention is not limited to the use of air as a transport medium of heat energy and humidity, but that, moreover, any other suitable gas, such as nitrogen, can also be used.

The process according to the invention is preferred for the purpose of changing the surface curvature of synthetic contact lenses used to correct the erroneous vision of a human eye by increasing or decreasing the lens' curvature. In the process, an essential advantage consists of the treatment can be done in the absence of the contact lens wearer.

The invention further comprises material removal systems suitable to execute the process steps mentioned above and to allow in the described manner the treatment of both synthetic as well as natural materials, among them biological tissues. In these systems, means are provided with which a gas stream is passed over the point of removal during the effect of the laser energy, said gas stream having a prescribed temperature, relative humidity and/or flow velocity as it flows over the point of removal. Preferred gas means include air, but other gases are also suitable, such as nitrogen.

In an especially preferred embodiment, the systems are equipped with means to pre-select the temperature, the relative humidity and/or the flow velocity from prescribed value ranges. The selection can be made prior to the beginning of the removal process, with devices present to maintain the pre-selected values during the entire removal process.

Furthermore, the means or devices to pre-select or change the temperature, relative humidity and/or the flow velocity are coupled to a control circuit that, for example, issues control signals depending on the values prescribed and according to a temporal function. This control circuit can also be coupled to an air heater and/or to an air humidifier.

It is advantageous for the air humidifier to be equipped with a mister, preferably an ultrasound mister, that discharges moisture at a constant drop size of <4 μm. For example, this applies to refractive laser surgery using laser radiation at a wavelength of 193 nm, wherein the misting output should be 0.5 to 2 ml/min. This produces an optimum mist density that takes into account necessary moisturization while minimizing water condensation.

Furthermore, means are provided that influence the flow direction of the gas or of the air such that the ablation by-products do not pass through the laser beam cross section, thus preventing the radiation intensity from being influenced in indefinable ways. To this end, for example, two annular flow channels are provided around the laser beam arranged one after the other in the direction of the laser beam, one of which is equipped with discharge openings and the other is equipped with inlet openings for the air stream. In the process, the discharge openings of one of the two flow channels and the inlet openings of the other flow channel are positioned so that the air stream is directed essentially parallel to the laser beam, preferably with a flow direction opposite to the direction of the laser beam.

Depending on the application, it can also be an advantage if the direction of the incoming air makes an angle of 0-70° with the tangent at the point of removal, and if the inlet openings used to suction the air stream away from the point of removal are designed such that the direction of the exiting air makes an angle of between 0 and 70° with the tangent to the point of removal as well.

To determine the humidity value at the point of removal, a light scattering measurement device, for example, is provided in which the intensity of the reflection of a special laser beam directed at the material surface, the wavelength of which lies in the visible or infrared spectral range, is used as a measure of the humidity at the surface. The physical parameters of this special laser radiation, in particular intensity and wavelength, are selected to be compatible with the characteristics of the material to be treated such that no change occurs in the material characteristics as a result of this radiation. To measure the current temperatures at the surface of the material without contacting it during the removal process, a commercially available thermal camera can be provided.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a system to remove material with the help of laser radiation, in which a laser beam is directed at the material surface and a device to climatize the environment at the surface is provided to feed a climatized air stream.

FIG. 2 is an example of the embodiment of a feed and discharge device for a climatized air stream directed at the material surface and its arrangement in the vicinity of the point of removal.

FIG. 3 is one way to position measuring devices to determine the temperature and humidity values in the vicinity of the point of removal.

FIG. 4 is another possible embodiment of the device to feed and discharge a climatized air stream.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

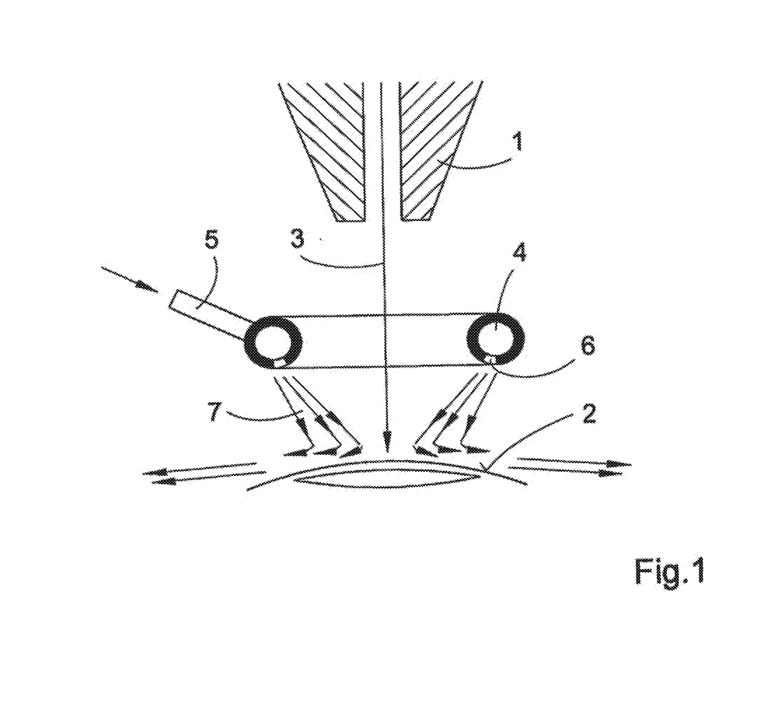

In FIG. 1, a tubular channel 1 is shown. A laser beam 3 exits the end of this channel and is directed at the surface of an object 2. A system of this type can, for example, be used to change the curvature of contact lenses or can be used for photorefractive keratotomy in which the curvature of the cornea of a human eye can be corrected by means of the effect of the laser radiation in removing the biological tissue of the cornea.

It is assumed that during the ablation process, an energy input into the tissue of approximately 0.5 watts occurs. In the process, a majority of the energy is used for the ablation, but a considerable portion is converted to undesirable thermal, acoustic and fluorescent energy. The thermal loss portions would mainly disrupt the removal conditions since it influences the tear film, i.e. the moisture on the cornea, and the hydration characteristics of the cornea itself.

In order to attain a defined removal rate and thus the possibility of a defined shape of the cornea surface, the objective of the invention should be to lessen or if possible entirely remove undesired influences on the ablation conditions, by keeping the climatic environmental conditions constant.

According to the invention, to this end a tubular channel 4 is provided that is connected to an air conveyor (not shown) via a connection fitting 5 and a connecting line connected to it (also not shown).

The air conveyor feeds air to the tubular flow channel 4, and this air exits the flow channel 4 through discharge openings 6.

Here, the discharge openings 6 are arranged such that the flow directions 7 of the discharging air, which make an acute angle with the laser beam 3, are directed toward the surface of the object 2.

Flow channel 4 is circular and arranged centrally around the laser beam 3, whereas the discharge openings 6 are distributed radially symmetric around the laser beam so that the flow directions 7 as a whole form approximately a circular cone surface. The laser beam 3 passes through the center of this cone surface.

The distance of the flow channel 4 to the object 2 is such that the peak of this circular cone surface coincides approximately with the point at which the laser beam 3 meets the object 2.

This results in the flow directions 7 meeting approximately at the point where the laser beam 3 meets the object 2 and counteracting one another such that the flow directions 7 reverse, with the flowing air being discharged radially outward. This results in the ablation by-products such as smoke and ablated tissue particles being collected by the air stream and discharged in the radial direction along with the air.

This results as much as possible in the ablation products not passing through the laser beam 3 and thus not being able to impair the intensity of the laser's radiation.

Also, according to the invention, the air conveyor is coupled to a climatization device for the air fed to the flow channel 4. The climatization device is designed such that the temperature and relative humidity of the air can be regulated. Also, means are present with which the temperature values, values for the relative humidity and also values for the amount of air fed per unit time can be pre-established prior to the beginning of the ablation process. To this end, both the air conveyor as well as the climatization device is equipped with means to enter commands, such as keys, switches or rotating knobs, which are part of a control system. Devices of this type for the purposes of air feed and climatization of the air, as well as corresponding input means are known from the state of the art and therefore do not need to be explained here in more detail.

If, for example, as a source for the laser radiation an Excimer laser, preferably an MEL 70 G-Scan, is provided that issues the very sensitive laser radiation having a wavelength of 193 nm, the invention can provide, by pre-selection of a temperature of 37° C., a relative humidity of approximately 100% and a flow velocity of approximately 0.5 m/s, that the climatic environmental conditions surrounding the point of removal during the ablation process are held constant within a relatively narrow range. This also ensures a relatively constant rate of removal, with the required precision being attained during shaping as much as possible.

In addition, both the air conveyors as well as the climatization devices can also be equipped with means to maintain the pre-selected values. Devices of this type that maintain the temperature, the humidity as well as the flow velocity of the air, are also known from the state of the art and are therefore not explained here in more detail.

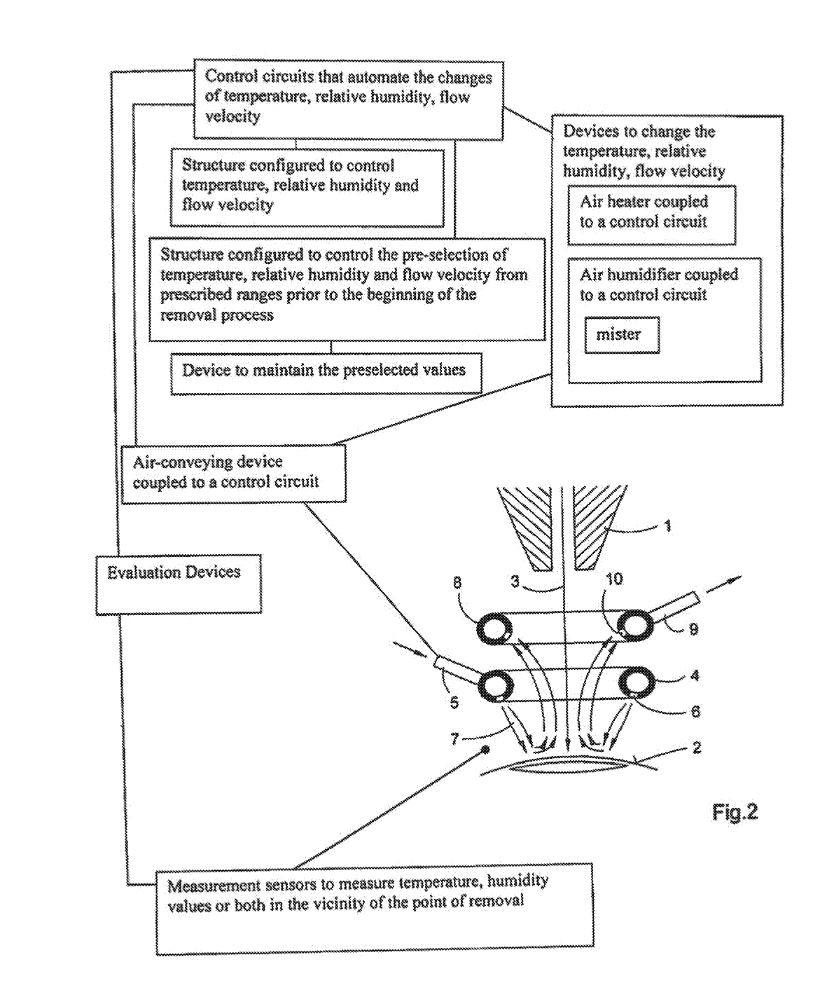

In an exemplary embodiment shown in FIG. 2 of the inventive system, in addition to the tubular flow channel 4, there is another tubular flow channel 8 provided that also encircles the laser beam 3 similar to flow channel 4, said channel 8 being located at a larger distance than flow channel 4 from the object 2, however. Also, in contrast to flow channel 4, it is not connected to an air feed device to feed air, but to a suction device (not shown in the drawing) that is connected to the flow channel 8 via a hose line (also not shown in the drawing) and via a connection fitting 9. Flow channel 8 has inlet openings 10 that are positioned essentially in the same arrangement as the discharge openings 6 in flow channel 4.

When this system is operated, an air stream is produced around the laser beam 3 that is first directed out from the discharge openings 6 of flow channel 4 toward the object 2 and then from the object 2 to the inlet openings 10 of flow channel 8.

In contrast to the embodiment variation according to FIG. 1, in this arrangement the ablation by-products are not discharged radially from the laser beam 3 outward, but (approximately in the opposite direction to the laser beam 3) are discharged through the inlet openings 10 into flow channel 8 and from there to the suction device. The result of this is that the ablation by-products (smoke, tissue particles) are not able to pass through the laser beam 3 and also do not contaminate the environment at the point of ablation or lead to odors endured by the person being treated.

In the process, the air exits the discharge openings 6 with a prescribed constant temperature, relative humidity and flow velocity and in this way provides for the defined climatic conditions at the object 2.

In contrast, in another embodiment of the system, instead of the temperature and humidity values of the air as well as its flow velocity being held constant during the removal process, measurement sensors can be provided to detect temperature and humidity values in the direct vicinity of the point of removal and for these sensors to be connected to the air conveyor and the climatization device via a control system.

This makes it possible to react to ongoing changes in the climatic environmental conditions very quickly by having the temperature of the flowing air or even its relative humidity increased or lowered based on the values detected so as to counteract the effect of the thermal dissipation within an even narrower range.

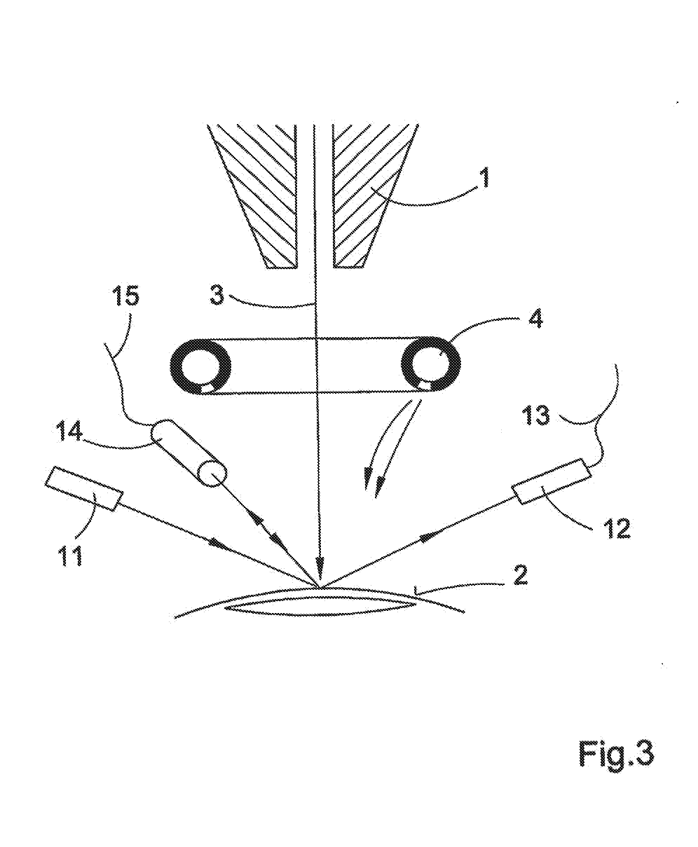

Examples for the arrangement of such measurement sensors are shown in FIG. 3. Here, for example, a light scattering measurement device is provided to measure the humidity value at the point of removal, said device consisting of a laser diode 11 that directs light in the visible or infrared spectral range at the object 2, and a photo detector 12 that receives the reflection of the laser radiation issued from the laser diode 11 and whose output signals are a measure of the humidity at the cornea surface.

The photodiode 12 is connected via a signal path 13 to the climatization device through an evaluation and control circuit (not shown).

The reflected scatter intensity of the laser radiation issued from the photo diode 11 essentially determines whether there is still a film of moisture present on the cornea surface or the extent to which it has already dried out.

To collect temperature values from the direct vicinity of the point of removal, a commercially available temperature meter can be used, such as a thermal camera, with its direction of measurement such that the temperature values are detected at the point of removal and are forwarded via a signal path 14 to the evaluation and control circuit that is connected to the climatization device.

This invention permits, in addition to the suctioning of the ablation by-products, a defined temperature and humidity to be established by means of a compact system in the direct vicinity of the treated location, for example of an eye being treated through photorefractive keratotomy. In this way the removal characteristics of the cornea tissue are held constant. As shown in detail, steady-state equilibrium of air humidity and temperature is established during the laser treatment by means of controlled feed and withdrawal of tempered, humidified air at defined flow velocity in the direct vicinity of the treatment location.

In a preferred embodiment, during the entire ablation process saturated air is used that is heated approximately to body temperature, and that has a relatively low flow velocity of approximately 0.5 m/s. A moisture film present on the object 2 before beginning the removal process first requires a low ablation rate. Since, however thermal energy is released during the ablation process, this leads to the moisture film increasingly drying out and as a result the ablation rate increasing. This is counteracted in the manner described.

When working with an Excimer laser as the radiation source for a wavelength of 193 nm, it should be noted that the absorption for this wavelength is considerably higher in water than in air or in another blanketing gas, such as nitrogen. Consequently, it is clearly necessary to counteract the tendency to dry out so as to maintain constant removal conditions. Insofar as this is concerned, the system according to the invention makes it possible to always establish temperature and humidity equilibrium between the object (contact lens or cornea) and the climatized environment at its surface. Variables such as an initially thick moisture film as well as increased drying out due to the energy input are compensated using the means proposed by the invention.

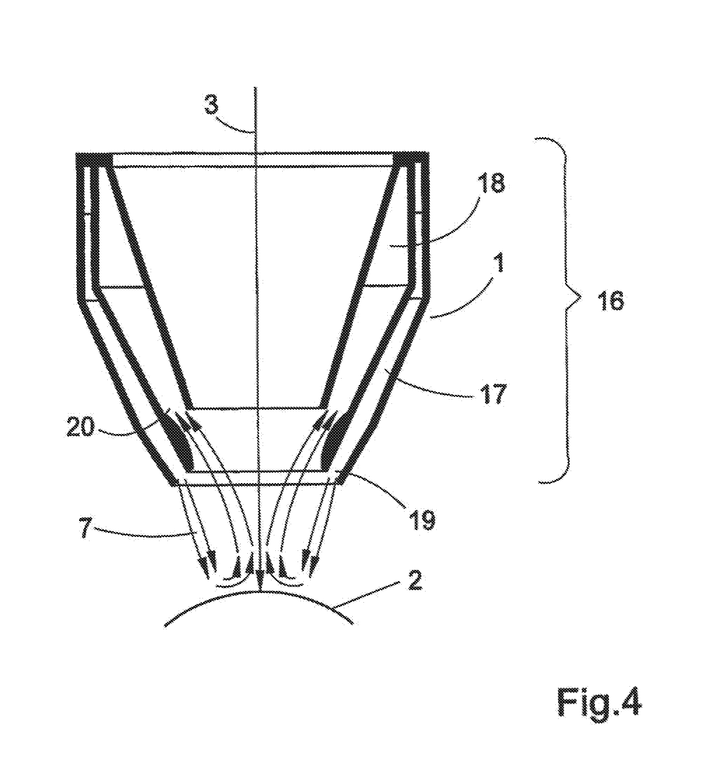

FIG. 4 shows another possible embodiment concerning the feed and withdrawal of a climatized air stream 7 directed toward and away from the surface of the object 2. As FIG. 4 shows, the end of the tubular channel 1 facing the object 2 has a conical section 16 with two chambers 17 and 18 that enclose the laser beam 3 concentrically. Chamber 17, which opens up into an annular discharge opening 19 is connected to an air climatization and conveying device (not shown in the drawing) that produces climatized air in chamber 17 at elevated pressure. The annular discharge opening 19 is designed such that the climatized air stream 7 discharged due to the overpressure is directed at the surface of the object 2 where it is reflected.

Chamber 18 is connected to a suction device (not shown in the drawing) that produces a reduced pressure. It has an annular inlet opening 20 through which the air stream 7 reflected by the surface of the object 2 is sucked and flows into the chamber 18 and is discharged to the suction device.

Hose lines can be provided to connect both chamber 17 to the air climatization and conveying device and to connect chamber 18 to the suction device, both of which are connected via connection fittings. The air climatization and conveying device and the suction device can be commercially available assemblies so that a more detailed explanation is not necessary here.

As already accomplished with the system according to FIG. 2, the embodiment according to FIG. 4 also permits the ablation products to be suctioned off without them passing through the laser beam 3 and thus impairing the intensity of the laser radiation. Because of the climatized air stream 7, the surface of the object 2 cannot dry out, resulting in uniform removal conditions being ensured.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. §112(f) are not to be invoked unless the specific terms “means for” or “step for” are recited in a claim.

1. (canceled)

2. An arrangement for ablation of material from a surface of an object by application of laser radiation, in a process of photorefractive keratectomy, comprising:
an air conveying device and a suction device guiding an air flow across an ablation site for a duration of an ablation process;
wherein a direction of air flowing towards the surface and a tangent to a surface at the ablation site intersect at an angle of 0° to 70°;
wherein a flow velocity of air flow is controlled to be in a range from 1 meter/second to 10 meter/second;
wherein a direction of the air flowing away from the surface and the tangent at the ablation site intersect at an angle of 0° to 70°;
wherein the flow velocity of the air flowing over the ablation site before a start of the ablation process is preselectable within a specified range, and
wherein the flow velocity is variable during the ablation process.

3. The arrangement as claimed in claim 2, the air conveying device comprising outlet openings and a suction device comprising entrance openings, and wherein air conveyance and definition of a flowing direction of the air is effected by two flow channels arranged consecutively in the direction of the laser beam and wherein the two flow channels are shaped as two rings centered around the laser beam, one of the two rings presenting the outlet openings and the other of the two rings presenting the entrance openings for the air flow.

4. The arrangement as claimed in claim 2, further comprising controls for preselecting a temperature, a relative air humidity or both the temperature and the relative air humidity within specified ranges before the start of the ablation process.

5. The arrangement as claimed in claim 2, further wherein the air conveying device keeps the air flowing across the ablation site during the ablation process with at least one quality selected from a group consisting of a temperature of minus 8° C.,-a relative air humidity of 80%, and a flow velocity of 3 meters/second.

6. The arrangement as claimed in any of claim 2, further comprising controls that vary the temperature and/or the relative air humidity within specified ranges during the ablation process.

7. The arrangement as claimed in claim 6, further comprising controls that vary the flow velocity within a range of 1 meter/second to 10 meters/second, an air heating device, controls for varying the temperature within a range of minus 20° C. to plus 30° C., an air humidifying device, controls that vary the relative humidity within a range of 0 to 100% or a combination of the foregoing.

8. The arrangement as claimed in claim 7, wherein the air humidifying device comprises a nebuliser that nebulises 0.5 ml to 2 ml of water per minute with a droplet size of <4 mm.

9. The arrangement as claimed in claim 6, further comprising control circuits that, during the ablation process, effect changes of temperature, relative humidity, flow velocity or a combination of the foregoing according to specified time schedule functions.

10. The arrangement as claimed in claim 7, further comprising control circuits that, during the ablation process, effect changes of temperature, relative humidity, flow velocity or a combination of the foregoing according to specified time schedule functions.

11. The arrangement as claimed in claim 8, further comprising control circuits that, during the ablation process, effect changes of temperature, relative humidity, flow velocity or a combination of the foregoing according to specified time schedule functions.

12. The arrangement as claimed in claim 6, further comprising sensors that measure temperature, humidity values or a combination of temperature and humidity values from the direct environment of the ablation site and, that via evaluation devices, are coupled with control circuits by which changes of temperature, relative humidity, flow velocity or a combination of the foregoing are effected as functions of the measured temperature, the humidity values or a combination of the temperature and the humidity values.

13. The arrangement as claimed in claim 7, further comprising sensors that measure temperature, humidity values or a combination of temperature and humidity values from the direct environment of the ablation site and, that via evaluation devices, are coupled with control circuits by which changes of temperature, relative humidity, flow velocity or a combination of the foregoing are effected as functions of the measured temperature, the humidity values or a combination of the temperature and the humidity values.

14. The arrangement as claimed in claim 8, further comprising sensors that measure temperature, humidity values or a combination of temperature and humidity values from the direct environment of the ablation site and, that via evaluation devices, are coupled with control circuits by which changes of temperature, relative humidity, flow velocity or a combination of the foregoing are effected as functions of the measured temperature, the humidity values or a combination of the temperature and the humidity values.

15. The arrangement as claimed in claim 9, further comprising sensors that measure temperature, humidity values or a combination of temperature and humidity values from the direct environment of the ablation site and, that via evaluation devices, are coupled with control circuits by which changes of temperature, relative humidity, flow velocity or a combination of the foregoing are effected as functions of the measured temperature, the humidity values or a combination of the temperature and the humidity values.

16. The arrangement as claimed in claim 10, further comprising, to measure humidity values, a straylight measuring device wherein an intensity of a reflection of a laser beam from the material surface is used as a measure of humidity, the laser beam emitting wavelengths in the visible or infrared spectral range.

17. The arrangement as claimed in claim 12, further comprising a thermal camera for the contactless measurement of current temperatures on the material surface.

18. The arrangement as claimed in claim 2, further comprising an excimer laser emitting laser emitting radiation at a wavelength of 193 nm as a source of ablation energy.

19. The arrangement as claimed in claim 3, wherein the two flow channels are separated from the surface of the object.
